(19) United States
(12) Patent Application Publication
(10) Pub. No.: US 2018/0211722 A1
(43) Pub. Date: Jul. 26, 2018

(54) INSULIN ACTIVITY MAP

(71) Applicant: BOSTON HEART DIAGNOSTICS CORPORATION, Framingham, MA (US)

(72) Inventors: Michael Dansinger, Framingham, MA (US); Michael Riel-Mehan, Louisville, CO (US); Julia Maddalena, Framingham, MA (US)

(21) Appl. No.: 15/877,726

(22) Filed: Jan. 23, 2018

Related U.S. Application Data
(60) Provisional application No. 62/449,798, filed on Jan. 24, 2017.

(51) Int. Cl. G16H 20/17; G06F 19/26; G16H 50/50

(52) U.S. Cl. CPC G16H 20/17; G16H 50/50; G06F 19/26

(57) ABSTRACT

An insulin activity map is a tool for assessing a patient's metabolic risk, designing a treatment plan, disseminating the information in a useful way to the patient, and encouraging the patient to remain on a treatment course. The insulin activity map combines a variety of inputs such as insulin sensitivity and insulin production to generate a patient-specific diagnosis and treatment assessment. The insulin activity map fills an important gap in prior art testing methods, providing diagnostic insights that improve clinician and patient understanding of the progression of diabetes, allows at-risk patients to more easily avoid developing diabetes, and helps diabetic and prediabetic patients manage and control the disease.

### CROSS-REFERENCE TO RELATED APPLICATION(S)

This application claims the benefit of and priority to U.S. Provisional Application Ser. No. 62/449,798, filed Jan. 24, 2017, the entire contents of which are incorporated by reference.

### FIELD OF THE INVENTION

The invention generally relates to clinical tools for diabetes prevention, treatment, and monitoring.

### BACKGROUND

Diabetes covers a number of conditions related to problems with insulin in the body. For example, diabetes can occur when not enough insulin is produced or when the body does not respond appropriately to insulin. Since there is no cure for diabetes, treatment generally involves managing the disease and symptoms.

Diabetes has become a major health concern around the world. People with diabetes are at higher risk of kidney failure, heart disease, stroke, and other serious ailments. Medical costs for a diabetic are about twice as high as for those without the disease.

Currently about 10% of people in the United States have diabetes, more than a quarter of which is undiagnosed. Additionally, about a third of Americans have prediabetes, meaning that their blood sugar is higher than normal but not yet classifiable as type 2 diabetes. Of those with prediabetes, 90% are undiagnosed. The prevalence of undiagnosed metabolic conditions poses a major problem in healthcare, because preventable or treatable disorders go unrecognized, often leading to further complications. Diagnosing these conditions earlier and initiating targeted treatment regimens would decrease mortality and morbidity, as well as save significant medical costs.

Even when diabetes or prediabetes is diagnosed, problems exist with transmitting the information to patients in a way that is helpful in encouraging them to manage the disease. That is because diabetes treatment generally includes lifestyle changes that the patient must make on his or her own. Those lifestyle changes include weight loss, healthy diet, and exercise. Often patients find it difficult to make these changes, and as a result their situation deteriorates or simply does not improve as well as it could have, resulting in the occurrence of otherwise preventable complications.

### SUMMARY

The present disclosure relates to tools for assessing a patient's metabolic risk, designing a treatment plan, disseminating the information in a useful way to the patient, and encouraging the patient to remain on a treatment course. The disclosure provides an insulin activity map that combines a variety of inputs and generates a treatment assessment for a patient. The map reports insulin sensitivity and insulin production in a way that enhances understanding of which treatments would be beneficial to a patient. The insulin activity map is a tool that fills an important gap in prior art testing methods. It goes beyond mere beta cell testing, adding a combination of fasting insulin and glucose measurements to provide a better insulin activity assessment that informs a patient-specific treatment plan. It provides diagnostic insights that improve clinician and patient understanding of the progression of diabetes, allows at-risk patients to more easily avoid developing diabetes, and helps diabetic and prediabetic patients manage and control the disease.

The insulin activity map is generally a graph or chart with areas to plot a patient's baseline insulin sensitivity and beta cell activity. The map provides regions corresponding to various levels of risk associated with the measurements. As a result of the uniquely configured display of the chart, a patient can easily observe where his or her insulin activity falls within the regions, determine metabolic risk, and decide which direction the insulin and beta cell measurements must go in order to improve. By tracking the progress of the patient's measurements over time, the patient gains a clearer understanding of the condition and is encouraged to continue making lifestyle changes for better health.

In certain aspects, the disclosure involves a method for determining a metabolic treatment for a patient. The method includes providing a diabetes status for the patient, providing an insulin sensitivity measurement for the patient, and providing a beta cell activity measurement for the patient. The method further includes determining a treatment for the patient based on a combination of the diabetes status, the insulin sensitivity measurement, and the beta cell activity measurement. In embodiments, the method further involves plotting the insulin sensitivity measurement and the beta cell activity measurement on a graph.

The diabetes status may be non-diabetic, prediabetic, diabetic, and diabetic with suboptimal control. Diabetes status may be determined using an A1c measurement, a glucose measurement, or a questionnaire. When A1c is used, an A1c measurement below 5.7 percent corresponds to non-diabetic, an A1c measurement from 5.7 and 6.4 corresponds to prediabetic, and an A1c measurement above 6.4 percent corresponds to diabetic. Additionally, an A1c measurement above 7 corresponds to diabetic with suboptimal control. The glucose measurement may involve a fasting blood sugar test, in which a blood sugar level less than 100 mg/dL corresponds to non-diabetic, a blood sugar level from 100 to 125 mg/dL corresponds to prediabetic, and a blood sugar level above than 125 mg/dL corresponds to diabetic. In other embodiments, the glucose measurement involves an oral glucose tolerance test, in which a blood sugar level less than 140 mg/dL corresponds to non-diabetic, a blood sugar level from 140 to 200 mg/dL corresponds to prediabetic, and a blood sugar level above than 200 mg/dL corresponds to diabetic.

In certain embodiments, one or both of insulin sensitivity and beta cell function is measured by homeostatic model assessment. Insulin sensitivity below 25% is very low insulin sensitivity, insulin sensitivity between 25 and 33% is low insulin sensitivity, insulin sensitivity between 34 and 50% is moderate insulin sensitivity, insulin sensitivity between 51 and 75% is borderline insulin sensitivity, and insulin sensitivity greater than 75% is normal insulin sensitivity. Beta cell activity below 25% is very low beta cell activity, beta cell activity between 25 and 40% is low beta cell activity, beta cell activity between 41 and 60% is moderately low beta cell activity, beta cell activity between 61 and 75% is borderline beta cell activity, beta cell activity between 76 and 149% is normal beta cell activity, beta cell activity between 150 and 200% is excessive beta cell activity, and beta cell activity greater than 200% is very excessive beta cell activity.

In embodiments, if insulin sensitivity is very low, low, moderate, or borderline, then the treatment includes a recommendation to consider weight loss and exercise. If insulin sensitivity is very low, low, or moderate and A1c is greater than 5.6, then the treatment further includes a recommendation to take metformin.

In embodiments, if beta cell activity is very low, low, moderately low, or borderline, then the treatment includes a recommendation to consider a therapy to reduce beta-cell demand. The therapy to reduce beta-cell demand may include carbohydrate reduction, GLP-1 agonists, and thiazolidinediones (TZD). If beta cell activity is very excessive, then the treatment includes a recommendation of carbohydrate reduction. If the diabetes status is diabetic and beta cell activity is low or very low, then the treatment includes insulin. If the diabetes status is diabetic with suboptimal control, then the treatment includes glucose-lowering medications.

### BRIEF DESCRIPTION OF THE DRAWINGS

FIG. 1 is an insulin activity map in accordance with the present invention.

FIG. 2 shows treatments correlated with various positions on an insulin activity map, also known as a metabolic risk map.

FIGS. 3 and 4 show examples of patient data plotted on the map.

FIG. 5 shows multiple points plotted on a map representing changes over a course of treatment.

FIGS. 6-9 show side-by-side comparisons of two patient maps.

FIG. 10 shows the proportion of patients in a sample population who fall into various sectors of the plot, both overall and by diabetes status.

FIGS. 11 and 12 show another insulin activity map according to the present invention.

### DETAILED DESCRIPTION

The insulin activity map is a useful tool for determining a treatment plan for a diabetic patient and monitoring the efficacy of the treatment over time. It provides a baseline assessment of the patient, given the patient's metabolic risk factors including insulin sensitivity and beta cell activity, which can be measured by a homeostatic model assessment (HOMA). For that reason, the insulin activity map can also be called a metabolic risk map. The map includes regions that correspond to various risk levels, which emanate out from a central region representing “normal” metabolic risk. Depending on where the patient's baseline measurements fall within the various ranges, a clinician can make a recommendation accordingly. Due to the unique design of the insulin activity map, the results are more easily interpreted and understood by the patient. Additionally, progress is more easily monitored because subsequent tests at regular time intervals throughout the course of treatment can be plotted on the chart and compared with the baseline. This allows both the clinician and patient to observe how the treatment regimen has affected the patient's condition. The insulin activity map and its uses will be more easily understood with reference to the figures and examples described below.

The insulin activity map takes advantage of glucose and insulin measurement methods known in the art. For example, the A1c test provides information about average levels of blood glucose over the past three months. The A1c test (sometimes called the hemoglobin A1c, HbA1c, or glycohemoglobin test) is based on the attachment of glucose to hemoglobin. Normal A1c levels are below 5.7 percent. Levels between 5.7 and 6.4 are prediabetic, and above 6.4 percent are diabetic. More information about A1c testing can be found in Sacks, 2012, Diabetes Care 35(12):2674-80. Other glucose tests are known in the art, and generally involve drawing a small amount of blood, often after a period of fasting. Oral glucose tolerance tests additionally involve consuming a measured amount of glucose and collecting additional blood samples at set intervals.

Other tests can be used with the present invention to determine insulin sensitivity and production. The homeostatic model assessment (HOMA), first described in 1985, is used to quantify insulin resistance and beta cell function. See Matthews et al., 1985, Diabetologica 28(7):412-19. Beta cell function represents insulin production. Its use in combination with insulin sensitivity gives a better overall assessment of the role of insulin in a patient.

Insulin may be measured in the same blood sample as used in the glucose measurement. It is common however to take an average of multiple samples drawn a few minutes apart since insulin secretion rises and falls over time.

The model shows the interaction between beta cell function and insulin resistance to compute steady-state insulin and glucose concentrations. These values are determined using the equations based on fasting glucose and insulin levels. HOMA relies on mathematical equations that describe the glucose regulation feedback loop. The formulae for beta cell activity (HOMA-% B) and insulin resistance (HOMA-IR) are given below, where glucose is in mg/dL and insulin is in μU/mL:

HOMA-% B=(360×Insulin)/(Glucose−63)

HOMA-IR=(glucose×insulin)/405

Insulin sensitivity (HOMA-% S) can be calculated by taking the inverse of the HOMA-IR measurement. That measurement, combined with HOMA-% B, which provides information about beta cell function in the pancreas, can give a general overview of insulin in the body. The relationship between these levels reflects the balance maintained by a feedback loop between the liver and beta cells. Computer programs are available that calibrate the model to more accurately reflect modern insulin assays. Additional information about HOMA can be found in Preumont et al., 2007, Diabetes Care 30:1187-92; and Wallace et al., 2004, Diabetes Care 27(6):1487-95.

The HOMA-% S and HOMA-% B values may fall into different ranges that indicate various clinically significant levels of sensitivity and function. For example, insulin sensitivity may be considered “very low” insulin sensitivity when it falls below 40%, 25%, 10%, or 5%, for example. Insulin sensitivity may be considered “low” when it is above the “very low” range, but is below another cutoff such as 50%, 40%, 33%, or 25%, for example. Insulin sensitivity may be considered “moderate” when it is above the “low” range, but is below another cutoff such as 75%, 60%, 50%, or 40%, for example. Insulin sensitivity may be considered “borderline” when it is above the “moderate” range, but is below another cutoff such as 90%, 80%, 75%, or 60%, for example. Insulin sensitivity may be considered “normal” when it is greater than the “moderate” range.

Likewise, beta cell activity may be considered “very low” when it is below, for example, 40%, 30%, 25%, 10%, or 5%. Beta cell activity may be considered “low” when it is above the “very low” range, but is below another cutoff such as 50%, 40%, or 30%. Beta cell activity may be considered “moderately low” when it is above the “low” range, but is below another cutoff such 75%, 60%, or 50%. Beta cell activity may be considered “borderline” when it is above the “moderately low” range, but is below another cutoff such as 80%, 75%, or 65%. Beta cell activity may be considered “normal” when it is above the “borderline” range, but is below another cutoff such as 175%, 150%, or 125%. Beta cell activity may be considered “excessive” when it is above the “normal” range, but is below another cutoff such as 220%, 200%, or 180%. Beta cell activity may be considered “very excessive” when it is above the “excessive” range.

These ranges are not required to be exact. Because these measure physiological phenomena that occur on a continuum, the ranges are merely meant as a helpful way to visualize and discuss the measurements. The ranges recited above are exemplary and should not be considered limiting. Those of ordinary skill in the art would recognize other useful ranges as well.

In one embodiment, the ranges are as follows. Insulin sensitivity below 25% is “very low” insulin sensitivity, insulin sensitivity between 25 and 33% is “low” insulin sensitivity, insulin sensitivity between 34 and 50% is “moderate” insulin sensitivity, insulin sensitivity between 51 and 75% is “borderline” insulin sensitivity, and insulin sensitivity greater than 75% is “normal” insulin sensitivity. Beta cell activity below 25% is “very low” beta cell activity, beta cell activity between 25 and 40% is “low” beta cell activity, beta cell activity between 41 and 60% is “moderately low” beta cell activity, beta cell activity between 61 and 75% is “borderline” beta cell activity, beta cell activity between 76 and 149% is “normal” beta cell activity, beta cell activity between 150 and 200% is “excessive” beta cell activity, and beta cell activity greater than 200% is “very excessive” beta cell activity.

The insulin activity map disclosed herein uses information provided by such algorithms and translates it onto a map that informs a treatment plan. Therapies for treating diabetes and prediabetes generally involve some combination of weight loss, diet, and exercise, as well as insulin treatment, and medications such as metformin, glucose-lowering medications, and receptor agonists such as thiazolidinediones (TZD) and GLP-1 agonists. Any of these and other known treatments can be applied based on the patient's map position. Specific treatments based on map position are described below, and others would be understood by those of skill in the art.

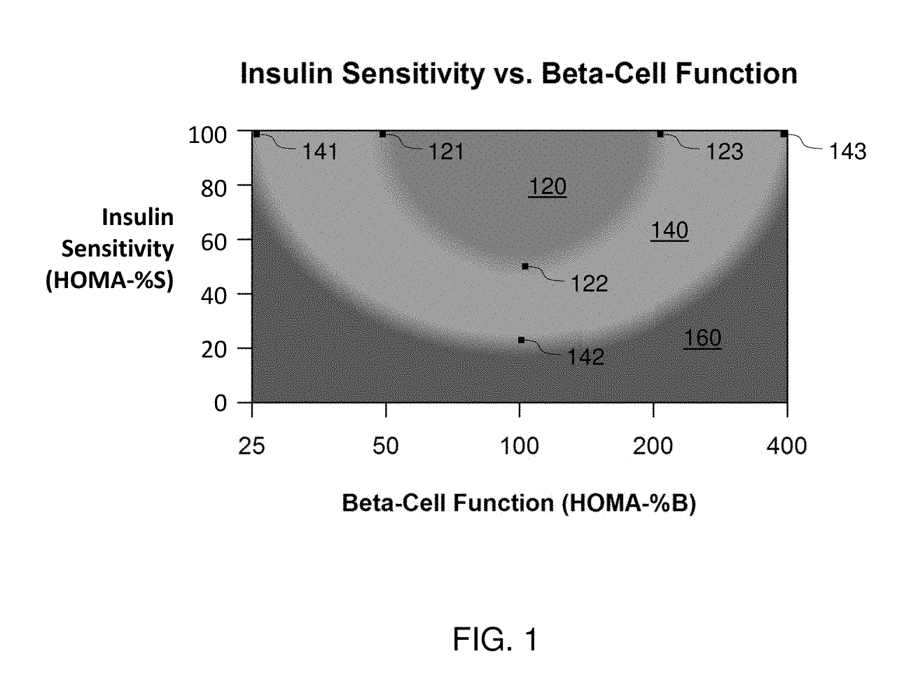

The insulin activity map takes the form of a sectional plot of HOMA-% B and HOMA-% S. The sectional plot is shown in FIG. 1. The plot is a clinically useful tool that provides important information that can be easily interpreted by clinicians and patients alike to formulate a baseline metabolic assessment, establish a treatment plan, and follow progress over time.

The plot of FIG. 1 includes insulin sensitivity (HOMA-% S) on the y-axis, ranging from 0 to 100%. The x-axis shows a logarithmic scale of beta cell function (HOMA-% B), with 100% at the midpoint of the scale. The left side of the scale is the range of beta cell activity between 25 and 100% and the right side is beta cell activity between 100 and 400%. The logarithmic scaling of the x-axis provides a more clinically meaningful and easily visualized map, which will aid in understanding and interpretation of the results.

The plot is divided generally into three regions representing various levels of metabolic risk. Region 120 is a semi-circle that approximately covers the region circumscribed by points 121, 122, and 123. Point 122 represents 50% insulin sensitivity and 100% beta cell activity; point 121 represents 100% insulin sensitivity and 50% beta cell activity; and point 123 represents 100% insulin sensitivity and 200% beta cell activity. Region 120 may be considered the region representing “normal” or low metabolic risk. In some embodiments of the plot, region 120 is colored green. Accordingly, region 120 may be referred to as the green region.

Region 140 is a larger semi-circle that surrounds the green region. The outer boundary of region 140 generally circumscribes points 141, 142, and 143. Point 142 represents 25% insulin sensitivity and 100% beta cell activity; point 141 represents 100% insulin sensitivity and 25% beta cell activity; and point 143 represents 100% insulin sensitivity and 400% beta cell activity. Region 140 may be considered the region representing borderline or moderate metabolic risk. In some embodiments of the plot, region 140 is colored yellow. Accordingly, region 140 may be referred to as the yellow region.

Region 160 occupies the remainder of the plot that is not covered by the green or yellow regions. Region 160 represents high metabolic risk. In some embodiments of the plot, region 160 is colored red. Accordingly, region 160 may be referred to as the red region.

The regions of the insulin activity map preferably do not have sharp boundaries, but rather have a gradient going from one section or color to the next. The gradient reinforces the fact that the boundaries are not precisely defined because the plot displays the metabolic risk associated with various factors, each of which exists on a physiological continuum.

To use the insulin activity map, a patient undergoes testing such as taking fasting glucose and fasting insulin measurements, and the results are plotted on the map. Testing may involve any of the tests described above, including glucose measurement, insulin sensitivity and production measurements, as well as questionnaires about lifestyle and medical history, weight, age, sex, body-mass index, etc. In a preferred embodiment, HOMA-% S and HOMA-% B values are calculated and plotted as a point on the map. The location of the point in relation to the three regions helps to establish a baseline that informs a treatment regimen.

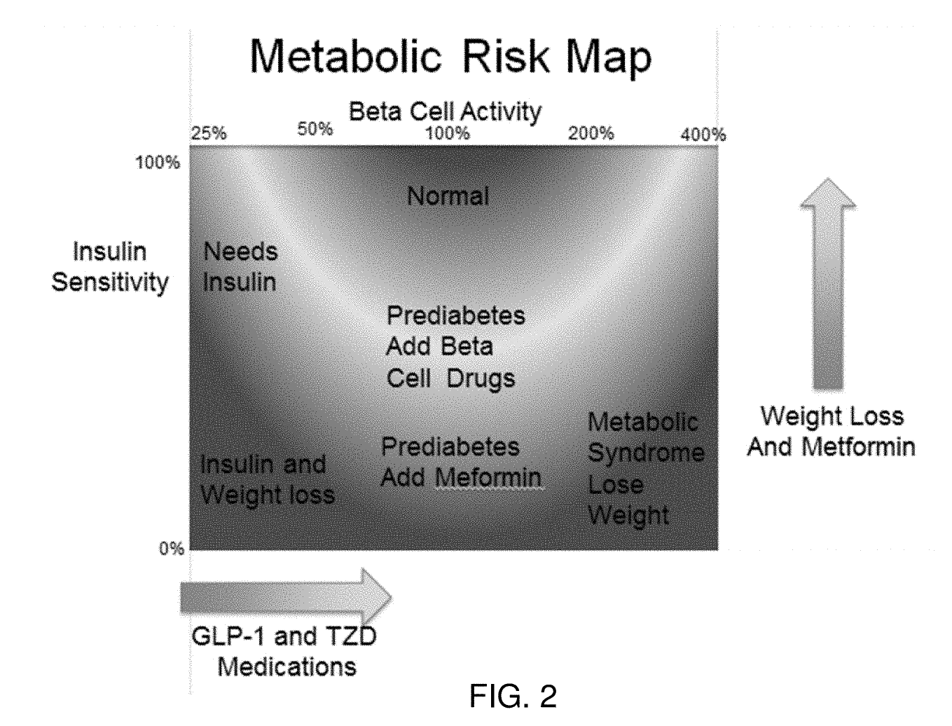

FIG. 2 shows how a patient's treatment plan may differ depending on where the plotted point falls on the map. For example, when beta cell activity is low and needs to be increased, GLP-1 agonists and TZD medications may be recommended. Likewise, to increase insulin sensitivity, the clinician may recommend weight loss, and/or metformin.

When a point is plotted in the areas on the left side of the map, insulin may be recommended. The lower parts of the map indicate a need for weight loss and/or metformin. Beta cell drugs may be indicated for patients showing prediabetes in the middle yellow portion of the map. Other permutations of treatments based on mapped locations would be evident to persons having ordinary skill in the art of diabetes monitoring and treatment.

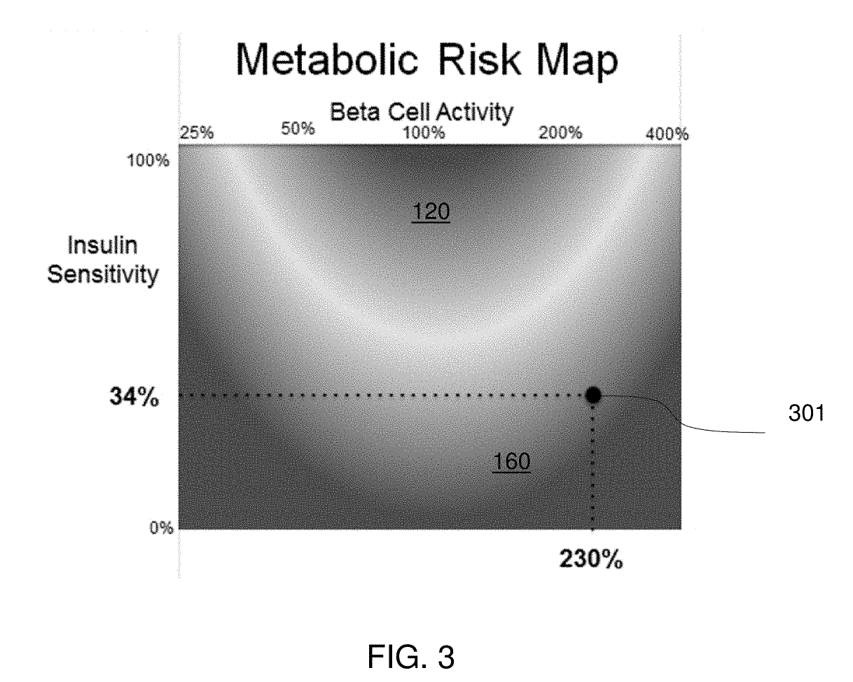

For example, FIG. 3 shows a point 301 plotted on the map for a patient with 34% insulin sensitivity and 230% beta cell activity. The point lies within the red region 160. For this particular patient, the clinician would interpret this insulin activity map plot to be high metabolic risk with low insulin sensitivity and excessive beta cell activity. The map provides an easily understood guide for getting those levels into better ranges. The treatment plan for the patient of FIG. 3 may be to consider weight loss and exercise combined with metformin.

The green region 120 serves as a target for subsequent tests. After following the prescribed treatment plan, subsequent testing allows another point to be placed on the map, from which the patient and clinician can observe whether the treatment has reduced beta cell activity and increased insulin sensitivity by a subsequent point plotted on the map.

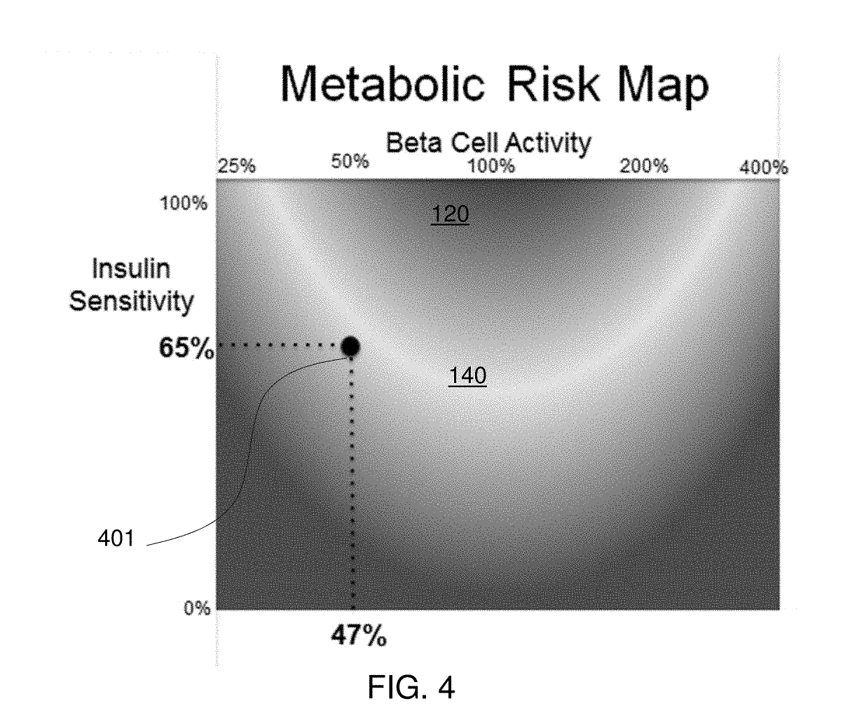

FIG. 4 provides another example, where the patient's data is represented by point 401 located is in the yellow region 140. The patient has 65% insulin sensitivity and 47% beta cell activity. In this case, the patient is prediabetic with a moderate progression rate, as determined by medical history. The clinician's assessment would be borderline insulin sensitivity with low beta cell activity. Because the patient would benefit from increased beta cell activity and increased insulin sensitivity, the treatment recommendation would be to consider weight loss, carbohydrate reduction, exercise, metformin, GLP-1 agonists, and/or TZD's.

The orientation and visual configuration of the plot makes it more conducive to clinical interpretation and understanding by a patient. The target of 100% insulin sensitivity and 100% beta cell activity is evident as the midpoint of the semicircles and an easily visualized goal. Patients can therefore easily see when subsequent testing has shown them to be closer or further away from that goal.

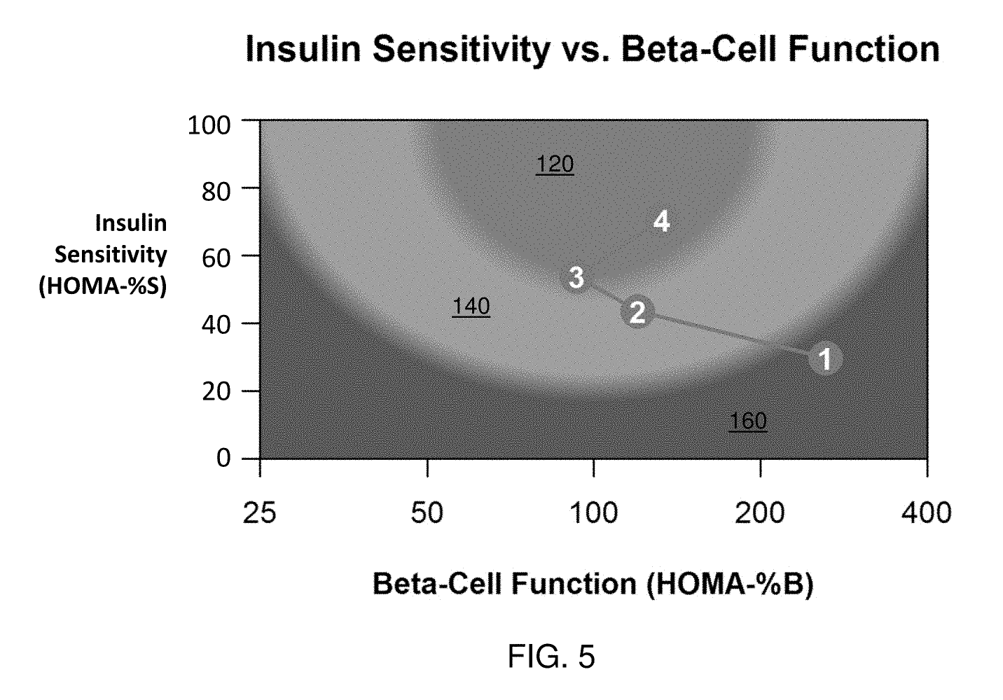

The baseline location on the plot provides important information about the patient's production and usage of insulin, and it informs the treatment plan going forward. The direction of movement over time provides important insights about their progress and ongoing opportunities for improvement and intervention. Multiple points plotted onto the map can provide these insights. For example, as shown in FIG. 5, a hypothetical patient is assessed for metabolic risk at t=0 and several more times throughout the course of treatment. In the first assessment (before treatment has begun) the patient is found to have approximately 250% beta cell function and 30% insulin sensitivity, and those measurements are represented on the chart as point 1. This serves as the baseline metabolic assessment for the patient. Based on the position of the baseline point 1, the clinician makes a recommendation, which in this case may include weight loss, exercise, and metformin treatment. While on the prescribed treatment regimen, the patient may be examined additional times, and the results plotted on the map. In the example shown, point 2 represents t=3 months, point 3 represents t=6 months, and point 4 represents t=12 months. Shorter or longer time intervals may be chosen as well.

The path created by connecting the successive time points 1-4 makes the patient's progress easy to monitor for the clinician and patient alike. Ideally, as patients improve their lifestyle and body weight, they move into more favorable regions of the plot. This provides easily identifiable visual confirmation of progress and helps to encourage patients to stay on a treatment course. As can be seen in FIG. 5, this patient's condition improved from red 160, to yellow 140, and eventually to green 120, over the course of a successful treatment designed according to the present invention.

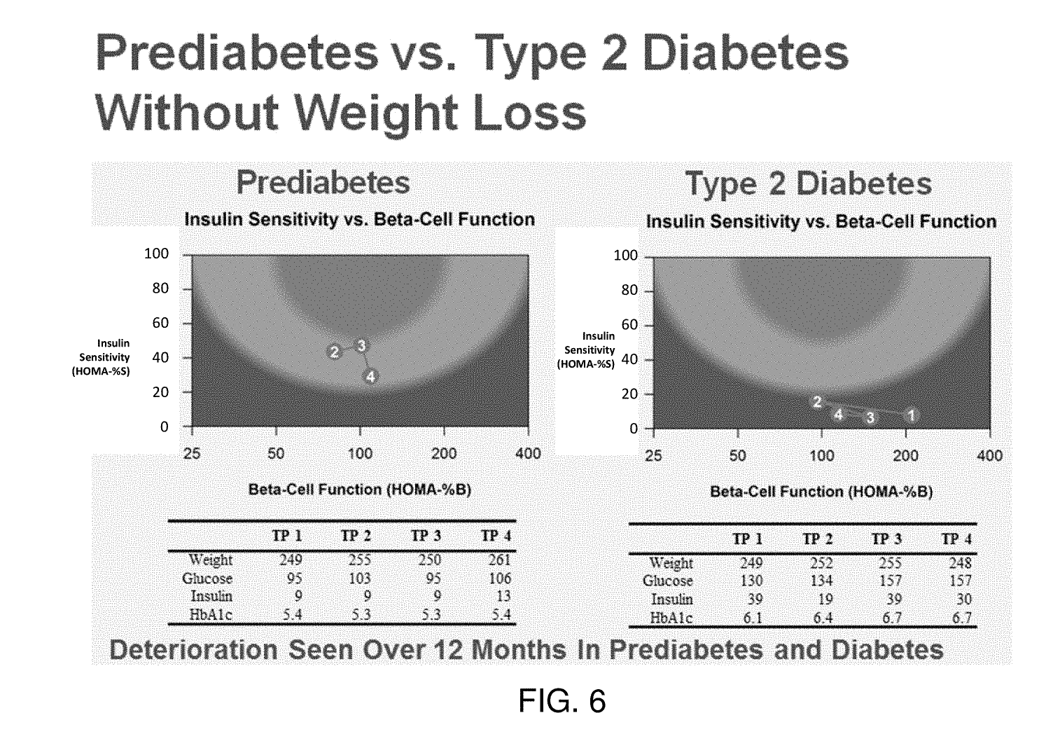

The insulin activity maps can be used to identify trends for an individual patient, and they can be used to compare how different factors among patients may lead to different outcomes or elucidate particular treatment recommendations. FIGS. 6-9 each show side-by-side comparisons of two patient maps to illustrate a particular point that can be revealed using the insulin activity maps. These comparisons highlight the clinical utility of the emerging mapping approach.

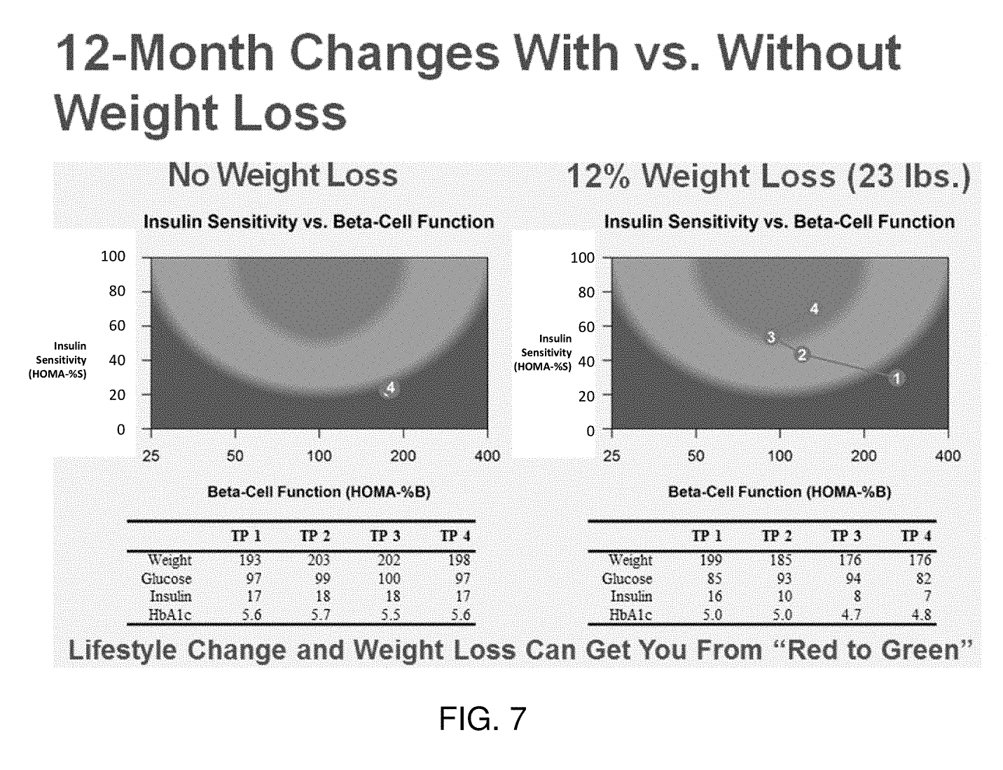

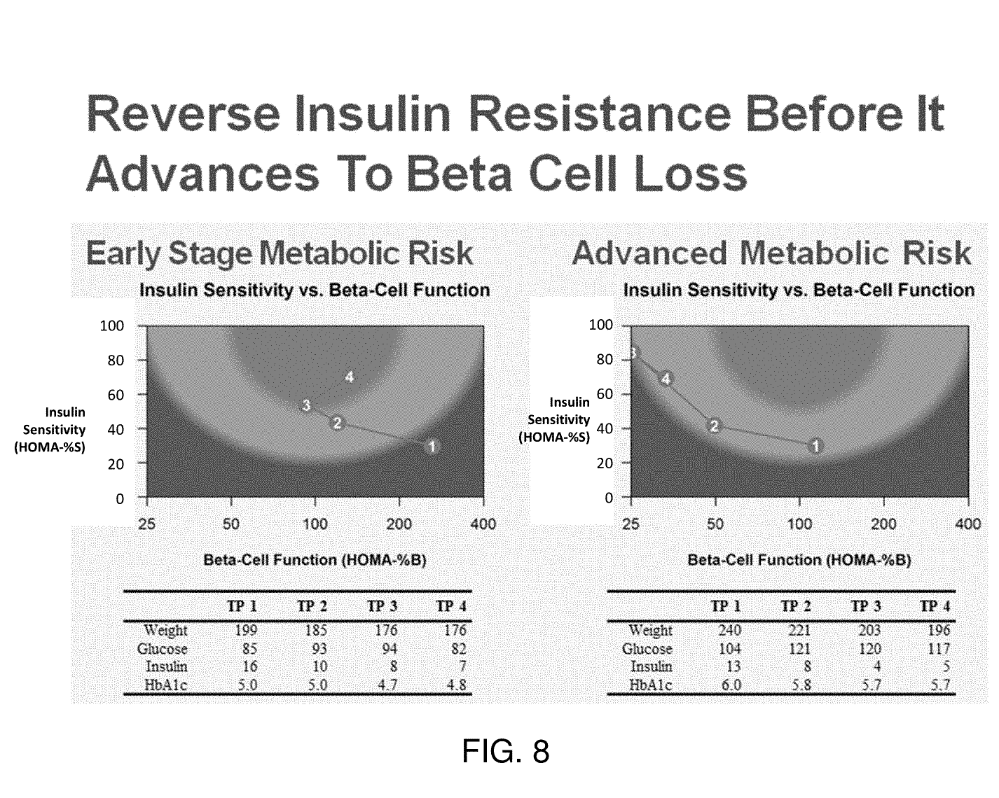

For example, FIG. 6 compares a patient with prediabetes to a patient with Type-2 diabetes over 12 months. The comparison shows how the deterioration of their conditions compares without weight loss. FIG. 7 compares the changes of two patients to show the difference that weight loss can make. The patient on the left did not lose weight (and in fact gained 5 pounds), whereas the patient on the right had steady weight loss, dropping about 12% of body weight over the course of the year. The two patients, whose baseline numbers were similar, showed significantly different results based on the factor of weight loss. The condition of the patient on the left remained mostly unchanged, staying in the red region; whereas the patient on the right moved from red to green. FIG. 8 compares a patient with early stage metabolic risk to a patient with advanced metabolic risk, based on beta cell loss.

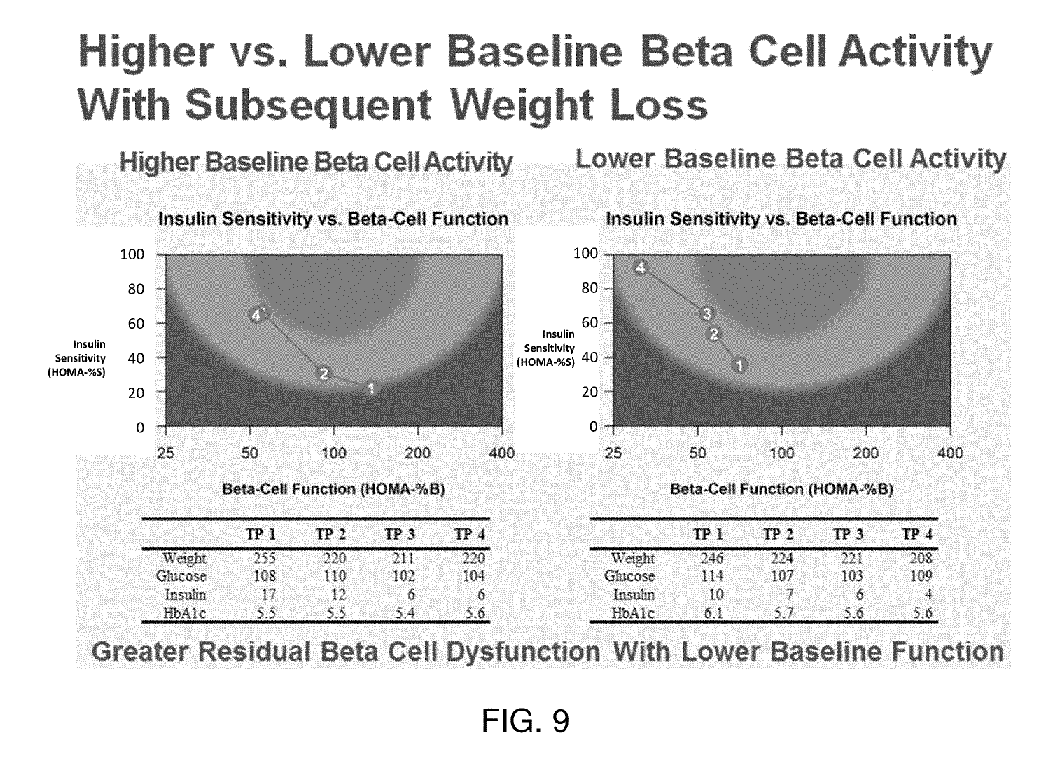

FIG. 9 compares two patients with different baseline levels of beta cell activity, and the effect of weight loss on their conditions. The patient on the left, with higher baseline beta cell activity moved closer to the green region, whereas the patient on the right stayed firmly within the yellow region. This result shows that there is greater residual beta cell dysfunction with lower baseline function. The particular design of the insulin activity map makes such observations possible.

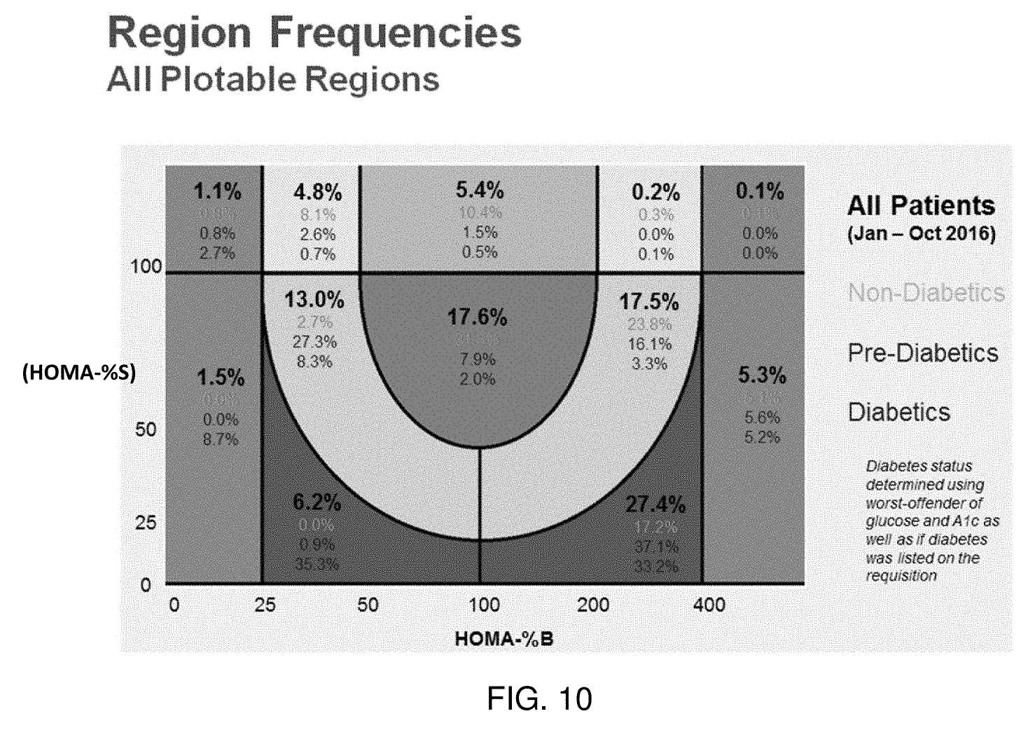

FIG. 10 shows the proportion of patients in a sample population who fall into various sectors of the plot, both overall and by diabetes status. The map in FIG. 10 extends from 0 to above 400 on the x-axis, and from 0 to above 100 on the y-axis, showing that certain patients fall outside the parameters of the chart as described in FIG. 1.

Although the plots thus far have shown particular values for the boundaries of the various regions, the regions can be shifted as needed. In other embodiments, for example, the yellow semicircle circumscribes the point at 100% insulin sensitivity and 300% beta cell activity, rather than the point at 100% insulin sensitivity and 400% beta cell activity. It should be understood that the exact boundaries of the regions are not meant to be precisely defined. They are meant to be used as a guide that illuminates for the observer which direction the patient needs to go to improve his condition (i.e., away from red, and towards green).

As shown in FIG. 10, the regions as defined here have about 23% of patients in the green, about 35% in the yellow, and about 42% in the red. There is an implicit subjective underpinning for the size of each category. Presently, insulin resistance and metabolic syndrome are at epidemic proportions, and the “normal” region representing less than a quarter of patients reinforces the prevalence of diabetics and prediabetics and clinically validates the zones as depicted. But as stated above, the regions of the chart describe the intersection of two physiological continua, not precisely demarcated boundaries. Importantly, regardless of the precise locations of the regions, the chart functions to direct the patient towards the optimal ends of the continua.

It is also important to note that, given the chart boundaries used in the maps heretofore described, about 11% of patients in the sample population are off the chart due to insulin sensitivity that is greater than 100%. By stopping at 100% insulin sensitivity, the design of the map minimizes confusion and maximizes clinical interpretation for the vast majority of patients. Another 5-6% of patients in the sample population are off the chart due to beta cell activity above 400%. Other similar displays, such as that shown in FIG. 10, can be used to include 100% of patients.

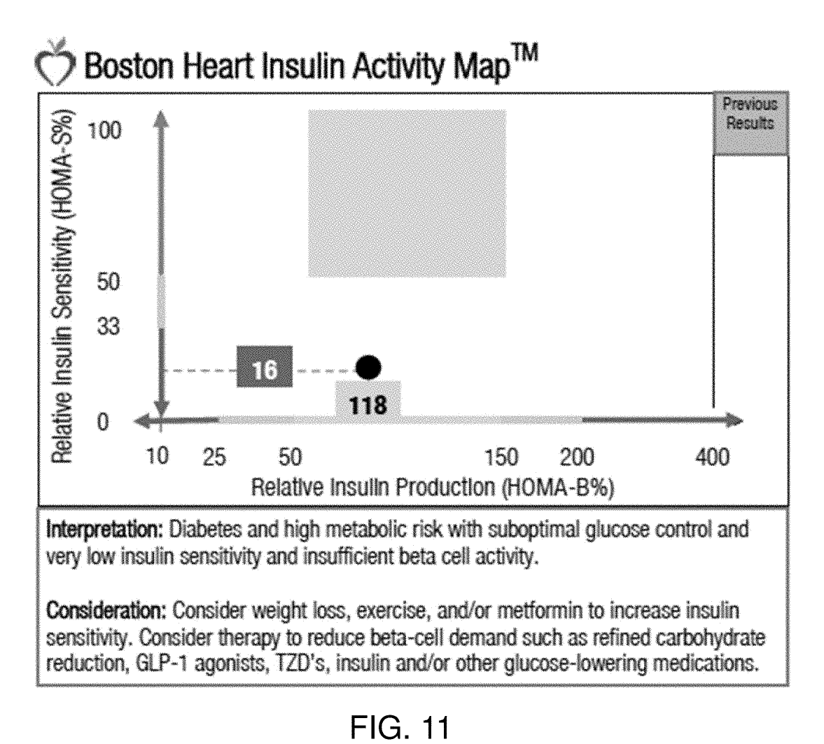

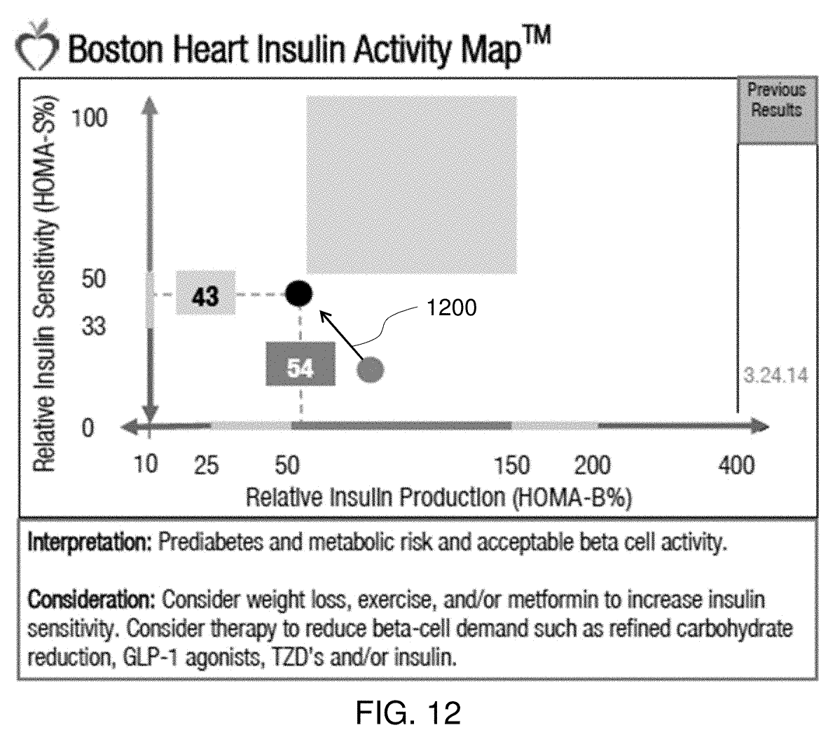

FIGS. 11 and 12 show a different embodiment of an insulin activity map, according to the present invention. According to this embodiment of the insulin activity map, low metabolic risk corresponds to HOMA-S % greater than 50 and HOMA-B % between 50 and 150, as indicated by the box in the middle of the map. In this embodiment of the insulin activity map, HOMA-S % between 33 and 50 is considered moderate or borderline metabolic risk; and HOMA-B % between 25 and 50, or between 150 and 200, is considered moderate or borderline metabolic risk. High metabolic risk for HOMA-S % is below 33; and high metabolic risk for HOMA-B % is below 25 or above 200.

FIGS. 11 and 12 show data mapped onto this version of the insulin activity map, from a test patient taken at two different points in time. FIG. 11 shows the results of a first test, and FIG. 12 shows the results after a course of treatment that was determined based on the patient's location on the insulin activity map of FIG. 11. The progress from FIG. 11 to FIG. 12 shows the usefulness of the insulin activity map for diagnosing a patient, determining his metabolic risk, designing a treatment plan, and monitoring progress over the course of the treatment plan.

In FIG. 11, test results show that the patient had a HOMA-B % of 118 and a HOMA-S % of 16. The HOMA-B % reading is in the normal range (i.e., between 50 and 150), but the HOMA-S % reading is low (i.e., below 33) indicating a metabolic risk. The interpretation is that this patient has diabetes and high metabolic risk with suboptimal glucose control and very low insulin sensitivity and insufficient beta cell activity. As a result, a recommendation for this patient would be to consider weight loss, exercise, and/or metformin to increase insulin sensitivity. The patient may also be encouraged to consider therapy to reduce beta-cell demand such as refined carbohydrate reduction, GLP-1 agonists, TZD's, insulin, and/or other glucose-lowering medications.

After some time of implementing one or more of these lifestyle changes, the patient is tested again, and the results are displayed in a new insulin activity map shown in FIG. 12. At this second time point, the patient has a relative insulin sensitivity of 43 and a relative insulin production of 54. This second reading is plotted as a second point on the map. Arrow 1200 shows the movement from the first reading to the second reading. As a result of the lifestyle change and weight loss, the new result shows the patient is prediabetic, with some metabolic risk and acceptable beta cell activity. Based on this new insulin activity map data point, the physician may recommend the patient lose more weight, continue exercising, and/or take metaformin to increase insulin sensitivity. The patient may also consider therapy to reduce beta-cell demand such as refined carbohydrate reductions, GLP-1 agonists, TZD's and/or insulin.

Over the course of the patient's treatment, additional measurements can be taken to monitor the overall progress and make refinements to the recommended course of action. With each new measurement, a physician can assess whether the treatment has gotten the patient closer to the low metabolic risk zone, indicated by the box in the middle of the insulin activity map.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

Any and all references and citations to other documents, such as patents, patent applications, patent publications, journals, books, papers, and web contents, which have been made throughout this disclosure, are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

What is claimed is:

1. A method for determining a metabolic treatment for a patient, the method comprising:
providing a diabetes status for the patient;
providing an insulin sensitivity measurement for the patient;
providing a beta cell activity measurement for the patient; and
determining a treatment for the patient based on a combination of the diabetes status, the insulin sensitivity measurement, and the beta cell activity measurement.

2. The method of claim 1, further comprising plotting the insulin sensitivity measurement and the beta cell activity measurement on a graph.

3. The method of claim 2, wherein the diabetes status is selected from the group consisting of non-diabetic, prediabetic, and diabetic, and diabetic with suboptimal control.

4. The method of claim 3, wherein the diabetes status is determined using an A1c measurement, a glucose measurement, or a questionnaire.

5. The method of claim 4, wherein the A1c measurement below 5.7 percent corresponds to non-diabetic, an A1c measurement from 5.7 and 6.4 corresponds to prediabetic, and an A1c measurement above 6.4 percent corresponds to diabetic, and further wherein an A1c measurement above 7 corresponds to diabetic with suboptimal control.

6. The method of claim 4, wherein the glucose measurement comprises a fasting blood sugar and wherein a blood sugar level less than 100 mg/dL corresponds to non-diabetic, a blood sugar level from 100 to 125 mg/dL corresponds to prediabetic, and a blood sugar level above than 125 mg/dL corresponds to diabetic.

7. The method of claim 4, wherein the glucose measurement comprises a an oral glucose tolerance test and wherein a blood sugar level less than 140 mg/dL corresponds to non-diabetic, a blood sugar level from 140 to 200 mg/dL corresponds to prediabetic, and a blood sugar level above than 200 mg/dL corresponds to diabetic.

8. The method of claim 4, wherein insulin sensitivity is measured by homeostatic model assessment.

9. The method of claim 8, wherein insulin sensitivity below 25% is very low insulin sensitivity, insulin sensitivity between 25 and 33% is low insulin sensitivity, insulin sensitivity between 34 and 50% is moderate insulin sensitivity, insulin sensitivity between 51 and 75% is borderline insulin sensitivity, and insulin sensitivity greater than 75% is normal insulin sensitivity.

10. The method of claim 9, wherein beta cell activity is measured by homeostatic model assessment.

11. The method of claim 10, wherein beta cell activity below 25% is very low beta cell activity, beta cell activity between 25 and 40% is low beta cell activity, beta cell activity between 41 and 60% is moderately low beta cell activity, beta cell activity between 61 and 75% is borderline beta cell activity, beta cell activity between 76 and 149% is normal beta cell activity, beta cell activity between 150 and 200% is excessive beta cell activity, and beta cell activity greater than 200% is very excessive beta cell activity.

12. The method of claim 11, wherein if insulin sensitivity is very low, low, moderate, or borderline, then the treatment includes a recommendation to consider weight loss and exercise.

13. The method of claim 12, further wherein if insulin sensitivity is very low, low, or moderate and A1c is greater than 5.6, then the treatment further includes a recommendation to take metformin.

14. The method of claim 13, wherein if beta cell activity is very low, low, moderately low, or borderline, then the treatment includes a recommendation to consider a therapy to reduce beta-cell demand.

15. The method of claim 14, wherein the therapy to reduce beta-cell demand is selected from the group consisting of carbohydrate reduction, GLP-1 agonists, and thiazolidinediones (TZD).

16. The method of claim 13, wherein if beta cell activity is very excessive, then the treatment includes a recommendation of carbohydrate reduction.

17. The method of claim 13, wherein if the diabetes status is diabetic and beta cell activity is low or very low, then the treatment includes insulin.

18. The method of claim 17, wherein if the diabetes status is diabetic with suboptimal control, then the treatment includes glucose-lowering medications.
